(19) United States
(12) Patent Application Publication
(10) Pub. No.: US 2023/0001205 A1
(43) Pub. Date: Jan. 5, 2023

(54) MUSCLE STIMULATION SYSTEM AND METHOD

(71) Applicant: EXCITA MEDICAL LTD., Yokneam (IL)

(72) Inventors: Jonathan APRASOFF, Kfar-Saba (IL); Giora ARBEL, Tel Mond (IL); Mordechay ESH, Givatayim (IL)

(21) Appl. No.: 17/784,655

(22) PCT Filed: Dec. 13, 2020
(86) PCT No.: PCT/IL2020/051284

§ 371 Date: Jun. 12, 2022

Related U.S. Application Data
(60) Provisional application No. 62/947,006, filed on Dec. 12, 2019.

(51) Int. Cl. A61N 1/36; A61N 1/04; A61F 7/00

(52) U.S. Cl. CPC A61N 1/36031; A61N 1/0452; A61N 1/36034; A61F 7/00; A61N 1/0492

(57) ABSTRACT

A system for stimulating a muscle of a user, the system comprising: a stimulating unit comprising (i) at least two electrodes configured to be placed in electrical contact with skin of a user in a vicinity of the muscle, and (ii) a pulse generator configured to generate an electrical pulse for application to the muscle through the at least two electrodes; a sensing unit comprising at least one sensor configured to measure a physiological parameter of the user and generate a sensing signal in response thereto; and a control processor operatively coupled to the stimulating unit and to the sensing unit and configured to receive the sensing signal from the at least one sensor and change a parameter of the electrical pulse applied by the at least two electrodes in response to the sensing signal.

### CROSS-REFERENCE TO RELATED APPLICATIONS

This application claims the benefit of priority of U.S. Provisional Patent Application No. 62/947,006 filed Dec. 12, 2019, the content of which is incorporated herein by reference in its entirety.

### FIELD OF THE INVENTION

Applications of the present invention relate generally to medical devices and specifically to system and methods for Electrical Muscle Stimulation (EMS).

### BACKGROUND

Electrical Muscle Stimulation (EMS) is the elicitation of muscle contraction using electric pulses.

In some cases, Electrical Muscle Stimulation (EMS) is employed during a physical workout, involving stimulating muscle contractions during a training session by delivering electrical pulses that stimulate and heighten muscle contraction, thereby intensifying the workout. Typically, EMS training strengthens core muscles and assists in building body mass and toning of body.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, device and methods which are meant to be exemplary and illustrative, not limiting in scope.

In accordance with some applications of the present invention, a system for stimulating a muscle of a user is provided. In some applications, the system delivers stimulation, e.g., electrical stimulation, to a target muscle, to elicit and intensify contraction of the muscle. For example, the electrical muscle stimulation (EMS) delivered by the system in accordance with some applications of the present invention, is used in sports training EMS devices, and/or for physical rehabilitation following an injury, and/or pain management in cases of muscle injury.

In accordance with some applications of the present invention, the system is an automatically adaptive muscle stimulation system comprising: (a) a stimulating unit comprising (1) at least one electrode (e.g., at least one pair of electrodes) configured to be place in contact with skin of the user to apply a stimulating current transcutaneously to a subcutaneous target muscle tissue of the user, and (2) a pulse generator configured to generate pulses to drive the electrode to apply the stimulating current to the tissue; (b) a sensing unit comprising at least one sensor and configured to detect a physiological parameter of the user and/or detect a muscle response, and generate a sensing signal in response thereto; and (c) a control processor unit operably coupled to and in communication with the stimulating unit and the sensing unit and configured to receive the sensing signal from the sensing unit to change, based on the sensing signal, at least one parameter of the stimulating current applied to the target muscle tissue.

Typically, the stimulating unit comprises at least two electrodes configured to be placed in contact with skin of the user in a vicinity of the muscle to be stimulated. For example, the muscles stimulated may include abdominal muscles, e.g., the abdominal rectus muscle and/or the external abdominal oblique muscle. For such applications, the electrodes are placed on an abdomen of the user in proximity to the abdominal muscles such that the electrical stimulation is delivered transcutaneously to the abdominal muscles. It is noted that the electrical stimulation may be delivered to a portion of one or more muscles rather than to the entire muscle. For some applications, the system comprises a pulse generator configured to generate an electrical pulse that is applied to the muscle through the at least two electrodes.

In accordance with some applications of the present invention, the system is configured to change or adjust application of the electrical pulse to the muscle in accordance with a physiological state of the user in response to a measured parameter of the user, to improve effectiveness of the electrical stimulation.

Accordingly, the system comprises one or more sensors configured to sense a physiological parameter of the user and to generate a sensing signal in response to the physiological parameter, and the control processor is configured to change a parameter of the stimulation applied by the stimulation unit in response to the sensing signal typically without active input from the user. For some applications, the physiological parameter is selected from the group consisting of: skin resistance, movements, body position, heart rate (HR) heart rate variability (HRV), oxygen saturation, acceleration, and a rate of respiration, and the sensor is configured to sense the physiological parameter selected from the group.

For example, the system is configured to synchronize application of the electrical pulse to the muscle with a phase of a respiratory cycle of the user. For some such applications, the system comprises at least one sensor for sensing breathing of the of the user, and the control processor is configured to receive input from the sensor and identify phases of a respiratory cycle. The control processor is further configured to synchronize the pulse generator in accordance with the respiratory cycle to generate the electrical pulse only during a pre-determined breathing phase, e.g., during inhalation or exhalation. Consequently, the electrical stimulation applied to the muscle of the user is applied during the inhalation phase, or during the exhalation phase.

It is hypothesized by the inventors that synchronizing application of the electrical pulse with a breathing phase of the user provides the muscle with the electrical pulse at an optimal stage of the respiratory cycle resulting in enhanced muscle contractions, leading to effective fat burn and improved muscle strength.

For some applications, the sensor for sensing breathing of the of the user comprises a piezoelectric element for sensing breathing of the user. Typically, the piezoelectric element is configured to generate a signal in accordance with variations in the girth of a thorax during the respiration cycle (for applications in which the piezoelectric element is positioned on a chest of the user).

Additionally, or alternatively the at least one sensor comprises a ECG sensor (e.g., a pair of electrodes) configured to measure ECG signals of the user and the control processor is configured to identify a phase of the respiratory cycle based on the input from the sensor and to drive the pulse generator to generate the pulse during the desired phase of the respiratory cycle, e.g., inhalation or exhalation.

In some applications, the pair of electrodes sense ECG signals of the user, and the control processor runs an algorithm to identify a respiration waveform based on the ECG-derived respiration information. In some applications, the control processor is configured to identify phases of the respiration cycle based on the EDR-based respiratory cycle and drive the pulse generator to apply the pulse through the electrodes during inhalation. It is hypothesized by the inventors that synchronization of the muscle stimulation timing with the ECG signal enables optimization of fat burn such that the muscle is stimulated while having optimal blood supply coming from the heartbeat phase.

Additionally, or alternatively, for some application, the stimulation unit further comprises a vacuum source for applying vacuum to the skin of the user in proximity of the muscles to be stimulated for increasing blood vessel dilation and blood flow leading to enhanced fat burn and energy consumption.

Additionally, or alternatively, for some applications, the stimulating unit comprises at least two accelerometers arranged in a predetermined pattern in relation to a center point of the muscle, and wherein each of the accelerometers is configured to measure movement of the muscle in one or more degrees-of-freedom.

For some application, the movement comprises at least one of: an onset, a modification, and a disappearance of a physical movement at the center point in reaction to the electrical pulse.

Further additionally or alternatively, a heat source is provided for heating superficial tissue during application of the EMS in accordance with some applications of the present invention. Typically, heating of the tissue in proximity to the electrically stimulated muscles increases metabolism in the tissue resulting in increased substance exchange between fat cells and blood vessels, increased rate of fat metabolic reaction in mitochondrion, and more effective fat burn (lipolysis). Additionally, since oxygen transport between blood vessels and muscle fibers increase with a rise in temperature, energy consumption by muscles is increased. Typically, increasing the superficial muscle temperature by about 20 degrees Celsius increases the rate of oxygen diffusion by more than 50%, resulting in more effective metabolism and fat burn.

For some applications, the stimulating unit comprises a plurality of electrodes arranged in an array and spatially distributed such that when placed in contact with the user, each electrode (or set of electrodes) stimulates a different portion of the target muscle tissue. Typically, the sensing unit is configured to sense a muscle response to detect muscle fatigue and the control processor is configured to discontinue application of stimulation to the fatigued portion of the muscle and initiate or increase stimulation to non-fatigued portions of the muscle, thereby maximizing muscle response and effectiveness of the EMS.

Thus, some applications of the present invention provide an enhanced EMS system and methods for maximizing the effectiveness of EMS for purposes of body shaping, muscle strengthening and fat burn. The system according to some applications of the present invention, combines heating of superficial body layers, dilation of blood vessels by application of superficial vacuum, and timing of the application of EMS pulses with breathing cycles and heart rate, to provide an enhanced and effective EMS treatment.

There is therefore provided in accordance with some applications of the present invention a system for stimulating a muscle of a user, the system including: a stimulating unit including (i) at least two electrodes configured to be placed in electrical contact with skin of a user in a vicinity of the muscle, and (ii) a pulse generator configured to generate an electrical pulse for application to the muscle through the at least two electrodes; a sensing unit including at least one sensor configured to measure a physiological parameter of the user and generate a sensing signal in response thereto; a control processor operatively coupled to the stimulating unit and to the sensing unit and configured to receive the sensing signal from the at least one sensor and change a parameter of the electrical pulse applied by the at least two electrodes in response to the sensing signal.

For some applications, the parameter of the electrical pulse includes a parameter selected from the group consisting of: an intensity of the pulse, a duration of the pulse and a shape of the pulse, or a combination thereof.

For some applications, the electrical pulse comprises a symmetric bi-phasic rectangular pulse shape comprising equal positive and negative pulse phases.

For some applications, the (i) a length of each of the pulse phases is in the range of 100-400 microseconds (μs), (ii) a length of an interphasic rest period is within the range of 40-100 μs, (ii) an amplitude of each of the pulse phases is in the range of 20-100 mA.

For some applications, the application comprises pulse bursts having a frequency of between 20-70 Hz and a duration of between 0.5-15 seconds.

For some applications, the physiological parameter of the user includes a parameter selected from the group consisting of: body movements, body position, heart rate (HR) heart rate variability (HRV), oxygen saturation, and a rate of respiration, and the at least one sensor is configured to measure the physiological parameter selected from the group.

For some applications, the sensor includes a sensor configured to detect breathing of the user and the control processor is configured to change the parameter of the electrical pulse in response to identifying a phase of the breathing cycle based on the sensing signal.

For some applications, the sensor includes a Galvanic Skin Response (GSR) sensor and the control processor is configured to determine a level of discomfort of the user based on the sensing signal from the GSR sensor, and calculate a correlation between the electrical pulse applied by the stimulating unit and the level of discomfort of the user and to change the parameter of the electrical pulse based on the correlation.

For some applications, the at least one sensor includes an accelerometer and the physiological parameter of the user includes contractions of the muscles measured using the accelerometer.

For some applications, the stimulating unit is configured to provide the electrical muscle stimulation such that contractions of the stimulated muscle exceed a desired threshold; and the control processor is configured to change the parameters of the pulse applied to the muscle in order to exceed the desired threshold of contractions, based on multiple uses of the system and: (i) iteratively varying the parameters of the electrical pulses, and (ii) measuring an effect of the varying of the parameters of the electrical pulses on the muscle contractions.

For some applications, the at least two electrodes include an array of at least 2-10 electrodes.

For some applications, a distance between two adjacent electrodes in the array is not greater than 2 cm.

For some applications, the control processor is configured to discontinue application of electrical pulses through a first one of the electrodes in the array and cause application of electrical pulses through a second one of the electrodes in the array in response to the sensing signal.

For some applications, the system further includes a heat source configured to heat the skin in proximity of the muscle to be stimulated by up to 20 degrees Celsius.

For some applications, the system further includes a vacuum source configured to apply vacuum to the skin of the user in proximity of the muscle to be stimulated.

There is further provided in accordance with some applications of the present invention a system for stimulating a muscle of a user, the system including: first and second electrodes configured to be placed in electrical contact with skin of a user in a vicinity of first and second portions of the muscle respectively; a sensor configured to detect a muscle response; and a control processor operably coupled to the first and second electrodes and to the sensor and configured to: cause the first electrode to apply stimulation to the first portion of the muscle; receive a detected sensing signal from the sensor in response to the stimulation applied by the first electrode indicative of at least one parameter of the muscle response of the first muscle portion; and based on the detected sensing signal: discontinue application of the stimulation through the first electrode to the first portion of the muscle; and cause the second electrode to apply the stimulation to the second portion of the muscle. For some applications, the at least one parameter of the muscle response includes a decrease in an intensity of contractions of the muscle.

For some applications, the sensor includes an accelerometer configured to sense muscle contractions.

For some applications, the first electrode includes a first subset of electrodes and the second electrode includes a second subset of electrodes.

For some applications, the control processor is configured to discontinue application of the stimulation through the first electrode when the at least one parameter exceeds a predetermined threshold.

There is yet further provided in accordance with some applications of the present invention a method for electrically stimulating a muscle of a user, the method including placing first and second electrodes in contact with skin in a vicinity of the muscle; stimulating a first portion of the muscle by applying electrical pulses to the muscle through the first electrode using a sensor, sensing a muscle response, the muscle response being indictive of a parameter of the muscle response of the first muscle portion; based on the sensing: discontinuing application of the electrical pulses to the muscle through the first electrode, and stimulating a second portion of the muscle by applying electrical pulses to the muscle through the second electrode.

For some applications, sensing a muscle response includes sensing a parameter of contractions of the muscle indicative of muscle fatigue.

For some applications, the parameter of the contractions includes an intensity of the contractions and the sensing includes sensing the intensity of the contractions.

For some applications, using the sensor includes using an accelerometer.

There is still further provided in accordance with some applications of the present invention a system for stimulating a muscle of a user, the system including: at least two electrodes configured to be placed in electrical contact with skin of a user in a vicinity of the muscle; a pulse generator configured to generate an electrical pulse for application to the muscle through the at least two electrodes; at least one sensor configured to sense breathing of the user, and generate a signal in response thereto; a control processor configured to receive the signal from the at least one sensor and synchronize the pulse generator in accordance with the signal to generate the electrical pulse only during inhalation.

For some applications, the at least one sensor includes a piezoelectric sensor.

For some applications, the at least one sensor includes an ECG sensor configured to sense an ECG signal of the user.

For some applications, the control processor is configured to run an algorithm to identify a respiration waveform by ECG-derived respiration information, and the control processor is further configured to identify phases of the respiration cycle based on the ECG-derived respiration information.

For some applications, the at least one sensor includes a first sensor and the system further includes a second sensor configured to sense one or more body parameters of the user and generate a signal in response thereto, the body parameter selected from the group consisting of: heartbeat rate, body movements, and body position; and the control processor is further configured to change the electrical pulse in accordance with the one of more body signals.

There is yet further provided in accordance with some applications of the present invention a method for electrically stimulating a muscle of a user using system including at least electrodes, the method including: placing the electrodes in contact with skin in a vicinity of the muscle; using a sensor, sensing breathing of the user; stimulating the muscle by applying electrical pulses to the muscle through the electrodes during inhalation.

For some applications, the method further includes heating the skin to a temperature of by up to 20 degrees Celsius.

For some applications, the method further includes applying vacuum to the skin.

There is still further provided, in an embodiment, a method for electrically stimulating a muscle of a user, the method comprising: placing a stimulating unit comprising (i) at least two electrodes configured to be placed in electrical contact with skin of a user in a vicinity of the muscle, and (ii) at least two accelerometers arranged in a predetermined pattern in relation to a center point of the muscle, wherein each of the accelerometers is configured to measure movement of the muscle in one or more degrees-of-freedom; operating a pulse generator to generate an electrical pulse for application to the muscle through the at least two electrodes; measuring, through the at least two accelerometers, movement of the muscle at the center point in response to the application; and adjusting, by a control processor operatively coupled to the stimulating unit and the pulse generator, at least one parameter of the electrical pulse, based, at least in part, on the measuring.

In some embodiments, the parameter of the electrical pulse is one or more of: an intensity of the pulse, a duration of the pulse, and a shape of the pulse, or a combination thereof.

In some embodiments, the method further comprises calculating, by the control processor, a normalized response value of the muscle to the electrical pulse, wherein the normalized response value represents a ratio between the movement of the muscle and a reference movement of the muscle before the application.

In some embodiments, the method further comprises estimating, by the control processor, a fatigue score of the muscle, wherein the fatigue score is based, at least in part, on the normalized response value calculated over time.

In some embodiments, the method further comprises modifying, by the control processor, a treatment protocol for a user, wherein the treatment protocol comprises a succession of electrical pulse applications, and wherein each of the applications is configured to generate a specified level of fatigue in the muscle.

In some embodiments, the modifying comprises modifying at least one of: an intensity of the electrical pulse application, a duration of the electrical pulse application, and a shape of a pulse signal of the electrical pulse.

The present invention will be more fully understood from the following detailed description of embodiments thereof, taken together with the drawings, in which:

### BRIEF DESCRIPTION OF THE DRAWINGS

Exemplary embodiments are illustrated in referenced figures. Dimensions of components and features shown in the figures are generally chosen for convenience and clarity of explanation and are not necessarily shown to scale. The figures are listed below.

FIG. 1 is a block diagram of the transmission of stimulation, information, and instructions, in the system for electrical muscle stimulation, in accordance with some applications of the present invention;

FIG. 2 is a graph modeling discomfort of the user during use of the system for electrical muscle stimulation, in accordance with some applications of the present invention.

FIG. 3A is a schematic illustration of an electrode array comprising a plurality of stimulating electrodes, in accordance with some applications of the present invention;

FIG. 3B is a schematic illustration of an electrode array comprising a plurality of stimulating electrodes placed in the vicinity of a target muscle, in accordance with some applications of the present invention;

FIG. 3C is a block diagram of system for electrical muscle stimulation comprising a demultiplexer, in accordance with some applications of the present invention;

FIGS. 3D-3E are flow charts representing first (FIG. 3D) and second (FIG. 3E) algorithm-based approaches for controlling selective application of the stimulation to different portions of the muscle, by selected subsets of electrodes in the electrode array, in accordance with some applications of the present invention;

FIG. 4 is a graph showing heart rate levels corresponding to stimulation applied to sedentary patients, in accordance with some applications of the present invention;

FIG. 5. is a schematic diagram of the functional steps in a process for calculating normalized muscle response to known EMS stimulation, in accordance with some applications of the present invention;

FIG. 6 is a schematic diagram of the functional steps in a process for muscle fatigue estimation, in accordance with some applications of the present invention; and

FIG. 7 illustrates an exemplary treatment signal pulse shape, in accordance with some applications of the present invention.

### DETAILED DESCRIPTION

Some aspects of the present invention provide a non-invasive ergonomic self-use system for electrical muscle stimulation (EMS), and methods of use thereof. The EMS in accordance with some aspects of the present invention, is used for various purposes such as physical rehabilitation, pain control, and in sports training EMS devices.

In some aspects, the system causes muscle contraction by applying electrical pulses to the muscles. In some aspects the pulses are generated by a pulse generator and applied through a plurality of electrodes positioned on skin in close proximity to the muscle to be stimulated and/or in close proximity to nerves innervating the muscle. Typically, the electrodes are placed in contact with the skin of a user and the electrical pulses are delivered transcutaneously to the underlying muscles. The system typically causes repeated contraction of the muscles by applying the pulsed electric current through the skin-placed electrodes.

In some aspects, a plurality of electrodes, e.g., 2-10 electrodes, e.g., 4-6 electrodes, are placed on skin in a vicinity of muscles to be stimulated. The electrical stimulation may be delivered to different muscles using different electrodes. Furthermore, electrical stimulation may be applied to different portions of a muscle. In some applications, the muscles stimulated may include abdominal muscles, e.g., the abdominal rectus muscle and/or the external abdominal oblique muscle. For such applications, the electrodes are placed on an abdomen of the user in proximity to the underlying abdominal muscles such that the electrical stimulation is delivered transcutaneously to the desired muscles. Additionally, or alternatively, the electrodes are placed on an arm, hip and/or buttocks of the user to stimulate underlying muscles.

In some aspects the pulses applied to the muscles may be triangular, square, rectangular, sinusoidal, partial sinusoidal, and/or sawtooth, etc.

In some aspects, the electrical pulses are synchronized and/or altered in response with body signals of the user. For example, the pulses are synchronized with a breathing cycle, heart rate—ECG reading, movements, and/or a body position of the user. For some such applications, the system comprises a sensor for sensing the body signals, and a control processor comprising processing circuitry for synchronizing the pulses that are applied to the muscles through the electrodes, in a manner that is based on the sensed body signals.

In some aspects the system comprises a sensor for sensing breathing of the user, and the electrical pulses are synchronized with the respiratory cycle such that the electrical pulses are generated based on the input from the sensor during a specific phase of the respiratory cycle. For example, the pulses are generated and applied to the muscle during the inhalation phase of the respiratory cycle.

In some aspects, synchronizing the stimulation pulses with the respiratory cycle and heartbeat of the user (systolic/diastolic cycles) provides optimization of oxygen consumption for maximum muscle effort, thereby maximizing training and fat burn. Typically, oxygen levels in the blood are controlled by the respiratory cycle and by the heartbeat cycle. Synchronizing between electrical stimulation of muscle and the level of oxygen in blood (that is fed to the muscles) is believed by the inventors to increase the level of muscle stress that can be achieved as well as energy consumption of the muscles. Unlike the natural process occurring when muscles are operated willingly by the user, proper timing with maximum oxygen level, as provided by use of the system in accordance with some applications of the present invention, can be externally controlled by synchronizing the muscles stimulation pulses with breathing and heart rate.

In some aspects, the system comprises a piezoelectric respiratory sensor in signal communication with the control processor and the pulse generator. The breathing cycle typically effects deformation of the piezoelectric respiratory sensor and a signal is generated by the sensor in response to the user's breathing. Typically, the control processor comprises circuitry configured to receive the signal from the respiratory sensor to determine when the onset of inhalation is occurring and cause the pulse generator to generate an appropriately timed pulse to the muscles through the electrodes, so that the system will stimulate the muscles during inhalation.

In some aspects the piezoelectric respiratory sensor (or any other sensor) is coupled to a garment or belt worn by the user. The garment or belt is typically placed across or around a body portion, e.g., a chest of the subject. Typically, deforming forces on the piezoelectric sensor in accordance with the respiratory cycle of the user, generates a signal indicative of the respiratory phase.

In some aspects the system further comprises a sensor for sensing additional body parameters of the user, such that application of the electrical stimulating pulses is additionally or alternatively changes in accordance with the body parameters. For example, application of the pulses is synchronized with a heartbeat rate of the user, oximeter readings, and/or motion of the user (e.g., as determined by an accelerometer).

In some aspects, the heartbeat rate and respiratory cycle information of the user is sensed by an ECG sensor. In some aspects, at least a pair of the electrodes placed on the skin of the user is configured for sensing ECG signals. Typically, the control processor is configured to identify phases of the respiration cycle based on the input from the ECG sensor and drive the pulse generator to apply the pulse through the stimulating unit during inhalation. In some aspects, the system comprises at least a first pair of electrodes configured to monitor ECG signals and a second pair of electrodes configured to apply electrical pulses to cause contraction of the muscles. In other words, according to an aspect of the invention the system comprises at least two electrodes for stimulating muscles by transmitting electrical pulses in synchronization with the respiration cycle of the user and a heartbeat rate of the user, and at least two electrodes monitor ECG signals in between transmitting the stimulating electrical pulses.

In some aspects of the present invention the system further comprises a vacuum source and the method additionally comprises applying vacuum to the skin to increase blood vessel dilation and blood perfusion in superficial layers of the body. Since superficial blood vessels in muscle tissue, skin and fat are not at full diameter, they may exhibit limited flow rates. Decreasing atmospheric pressure by applying vacuum typically increases blood vessel dilation and blood flow leading to enhanced fat burn and energy consumption.

In some aspects of the present invention, the system further comprises a heat source for heating the skin and superficial tissue of the user in the vicinity of the muscle designated for treatment thereby increasing metabolism and energy consumption contributing to fat burn and enhancing the EMS treatment. In some aspects one or more of the electrodes are heated to provide heating of the skin and superficial tissue.

In some aspects of the present invention a method is provided for non-invasively electrically stimulating muscles. In some aspects, the method comprises ramping up the electrical pulses to a passive pre-strained muscle. For example, training of a passive strained muscle can be performed by generating current pulses in specific sequences via at least first and second groups (e.g., arrays) of three or more electrodes placed in contact with the skin of the user. For such applications, the first group of electrodes stimulates the muscle to a specific level of strain and the second ramps up the stimulation to a tolerable level.

In some aspects the method further comprises measuring a parameter of the tissue as indication of effectiveness of the EMS treatment in accordance with some applications of the present invention. For example, effectiveness is measured by monitoring the muscle conductivity (via the stimulation electrodes, in time sequence with the stimulation pulses) and/or accelerometers (maximizing muscle movement) and/or piezoelectric sensors mounted in a belt (maximizing tension force), and/or calorie burn. Feedback may be displayed on a screen or monitor (e.g., by displaying the caloric expenditure).

In accordance with an alternative aspect of the present invention, the muscles are stimulated by High-Intensity Focused Electromagnetic (HIFEM) technology (indirect contact with the skin). For such applications, an applicator comprising a magnetic coil is placed on the skin at the treated area and stimulate the muscles by generating and inducing intensive focused electromagnetic field.

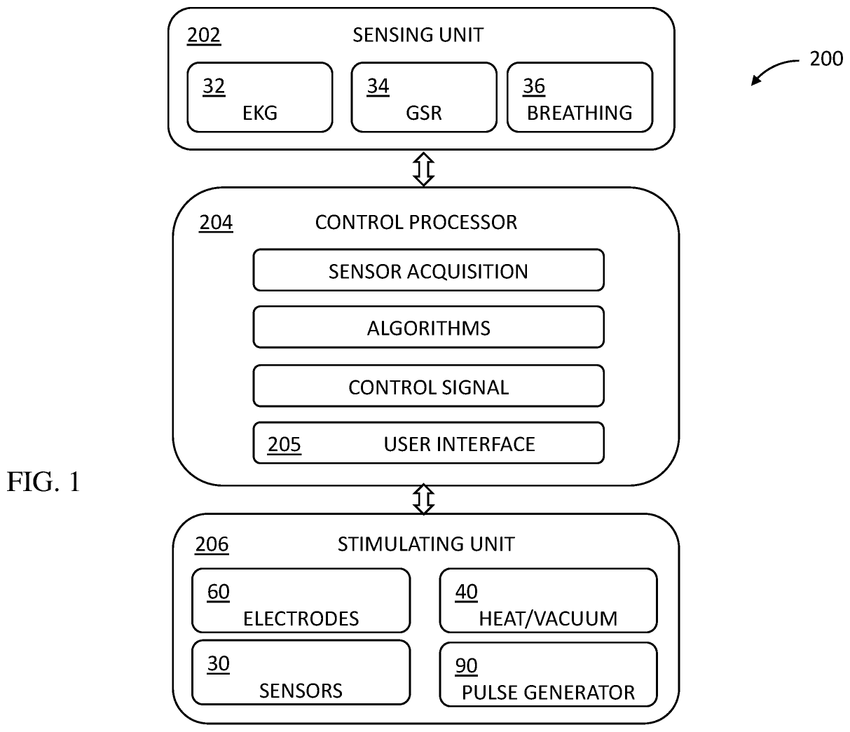

Reference is first made to FIG. 1, which is a block diagram of the transmission of stimulation, information, and instructions, in system 200 for electrical muscle stimulation, in accordance with some applications of the present invention.

As shown in FIG. 1, for some applications, system 200 comprises: (a) a stimulating unit 206 for applying stimulating pulses for stimulating muscles (and/or nerves innervating the muscles) of the user; (b) a sensing unit 202 for measuring physiological parameter of the user (e.g., mechanical, electrical and optical), and generating an output sensing signal in response to the measuring; and (c) a control processor unit 204 operably coupled to sensing unit 202 and to stimulating unit 206 and configured to use the output from sensing unit 202 to modulate stimulation parameters (e.g., the intensity and/or the pattern of stimulation, and/or duration of stimulation) applied to the user by stimulating unit 206.

Sensing unit 202 typically comprises one or more sensors 30 configured to sense a physiological parameter of the user, typically during a training session, and generate an output in response thereto. The output is typically further processed by a sensing processor such that a clean sensing signal is used to adjust of the stimulation based on the measured physiological parameter. For example, sensing unit 202 comprises one or more of the following sensors: an ECG sensor 32, a sensor 36 for detecting breathing of the user (e.g., a piezoelectric sensor), a Galvanic Skin Response (GSR) sensor 34, as shown in FIG. 1. Alternatively, or additionally, sensing unit 202 may comprise any other sensor, e.g., a sensor for measuring blood oxygenation and heart rate (e.g., a SPO2/PPG sensor), an Inertial Measurement Unit (IMU) sensor, and/or a motion sensor (e.g., an accelerometer).

The one or more sensors 30 may be coupled to the user using electrodes (e.g., in the case of the ECG and or the GSR sensors), and/or placed against the user's skin (e.g., as in the case of a PPG sensor), and/or coupled to a garment (e.g., as in the case of a piezoelectric sensor that measures chest movements of the user).

The output from sensing unit 202 is received by control processor 204 which comprises a sensing processor. Typically, the signal processor registers the output signal received from sensing unit 202 and in turn provides sensing unit 202 with power and/or control signals as needed (e.g. provides sensing unit 202 with defining parameters for operation such as sample rate and/or dynamic range). For some applications, the signal processor additionally comprises processing circuitry for processing the output signal from sensing unit 202 to derive parameters of the user that can be used by control processor 204 to control application of the stimulation to the user by stimulating unit 206. For example, the parameters of the user that can be derived from processing the output signal from the sensing unit are:
Heart rate—derived, for example, from the ECG, and/or PPG signals.
Heat Rate Variability (HRV)—derived, for example, from the ECG signal.
Heart-beat phase (systolic/diastolic)—derived, for example, from the ECG signal.
Breathing rate—derived, for example, from the piezoelectric sensor signal.
Respiratory cycle phase (inhalation/exhalation)—derived, for example, from the piezoelectric sensor signal.
Skin resistance as an indication of stress or discomfort—derived, for example, from the Galvanic Skin Response (GSR) sensor.
Blood oxygenation and heart rate—derived from the SPO2/PPG sensor, for example.
Muscle reaction to stimulation-derived, for example, from an Inertial Measurement Unit (IMU) sensor

Control processor 204 processes the signal from sensing unit 202 to generate a control signal that is received by stimulating unit 206 causing stimulating unit 206 to generate and apply stimulation to the user based on the instructions of control system 204. Thus, depending on the measured parameter by sensors 30 of sensing unit 202, a control signal is received by the pulse generator 90 of stimulating unit 206, which in turn generates pulses applied to the user via the electrodes and is configured to vary one or more stimulation pulse parameters depending on the control signal from control processor 202, without active input from the user.

Additionally, or alternatively, control processor 204 comprises a user interface 205 having a display such as a screen and/or at least one input means. User interface 205 allows the user, as well as a physician or a trainer, to view progress of the muscle stimulation and adjust the stimulation settings accordingly.

For some applications, control processor 204 and user interface 205 are configured such that the user (or a physician or a trainer) can select a muscle stimulation treatment protocol from a selection of predefined treatment protocols. The treatment protocol is typically selected according to the area of application of the stimulation, user physique and treatment goals. A treatment protocol may be defined by a time series of stimulation intensities and or patterns. Additionally, or alternatively, a treatment protocol may be defined by parameters such as a time series of a desired heart rate, comfort levels, and/or muscle activity. These parameters may be mapped onto the optimal stimulation series by control processor 204 in accordance with some applications of the present invention.

For some applications, control processor 204 is configured to assess calorie expenditure of the user. Typically, the calorie expenditure of the user is assessed based on statistic algorithms that use information derived from the measured physiological parameters of the user taken together with the intensity of the electrical stimulation applied to the user. For example, the output sensing signal generated by sensing unit 202 together with a known intensity of the estimation is used to calculate the caloric expenditure (kCal/min+total), and muscle work (kj/min) of the user. This information may be displayed to the user via user interface 205.

As described hereinabove, sensing unit 202 comprises a breathing sensor in accordance to some applications of the present invention. For some applications, control processor 204 is configured to identify specific phases in a breathing cycle based on the input from the breathing sensor. Typically, the phases of the breathing cycle are identified by an algorithm for detecting a breathing phase. The algorithm is based on a model that learns the user's breathing pattern morphologies using pattern recognition and machine learning tools in a manner that allows identification of the breathing phase regardless of background noise.

For some applications, control system 204 is configured to automatically (i.e., without user input) adjust at least one parameter of the muscle stimulation applied to the user in response to measuring physiological parameters of the user. Typically, control processor 204 comprises stimulation adaptive controller software configured to maximize a measured parameter and/or follow a predetermined time series of values of the measured parameter. For example, control processor 204 aims to maximize the mechanical contraction effect of the electrical muscle stimulation or follow a times series of heart rate values of the user. In order to ensure safety of the application of the electrical muscle stimulation, the stimulation adaptive controller software is configured to follow a set of limitations, e.g., limiting current of stimulation, rate of contractions (per time unit), level of discomfort, and/or maximal intensity of stimulation. Typically, in order to calculate and perform a safe and effective control of the stimulation, taking into account the training goal and the limitations, control processor 204 uses machine learning methods on the recorded signals (stimulations parameters, measured physiological signals, electrode sensor output) to create a mathematical model of the relationship between the stimulation parameters (e.g., frequency of pulse, shape of pulse, and/or timing of pulse) and the resulting muscle reaction (first order effects) and the greater effect of stimulation on discomfort and/or heart rate (second order effects).

The machine learning algorithm may be a non-linear regression for estimating a small number of parameters (e.g., 5-10 parameters) in a model-based algorithm or it may be a more complex neural network-based algorithm in a model-free estimation type of algorithm.

Once an estimation of a first and second order effect of the stimulation is generated, control processor 204 (e.g., using the adaptive controller software), is configured to use the inverse of such a model to calculate an optimal control protocol to ensure safety of the user together with maximal efficiency of the stimulation treatment. For some such applications, at the start of the stimulation treatment, control processor 204 goes into “fast learning mode” in which control processor 204 operates in two modes: (a) a high adaptivity mode in which the internal parameters that limit the extent of model adaption are set to allow a high level of change in a short time period; and (b) an exploratory stimulation mode. In the exploratory stimulation mode system 200 performs a series of stimulations (e.g., causes application of exploratory stimulation pulses to the muscles (e.g., of different intensity/duration/shape of pulses)) for learning the relationship between the applied pulse and the parameters of the subject measured by the sensors and processed by the control processor, thereby generating an optimal control protocol for each user.

Reference is still made to control processor 204. For some applications, control processor 204 is configured to assess a level of discomfort of the user resulting from the applied muscle stimulation. Typically, the level of discomfort is determined by a discomfort estimation algorithm that uses features derived from the physiological signals obtained from the user by the one or more sensors (e.g., heart rate, HRV, GSR, and/or breathing rate) to estimate the user's discomfort level. Control processor 204 is configured to change a parameter of the stimulation applied to the user based on the assessed level of discomfort (e.g., lower the intensity or duration of the simulation pulses or discontinue the stimulation. For some applications, control processor 204 is configured to maximize the stimulation that is applied to the user yet while not exceeding a maximal level of discomfort of the user. In such cases, control processor 204 generates an internal model of a reasonably expected discomfort level per a certain stimulation intensity. This model typically adapts over time given that discomfort levels generally decrease over time when a generally constant stimulation intensity is applied as the user grows accustomed to the stimulation and the muscle's response is reduced due to localized muscle fatigue.

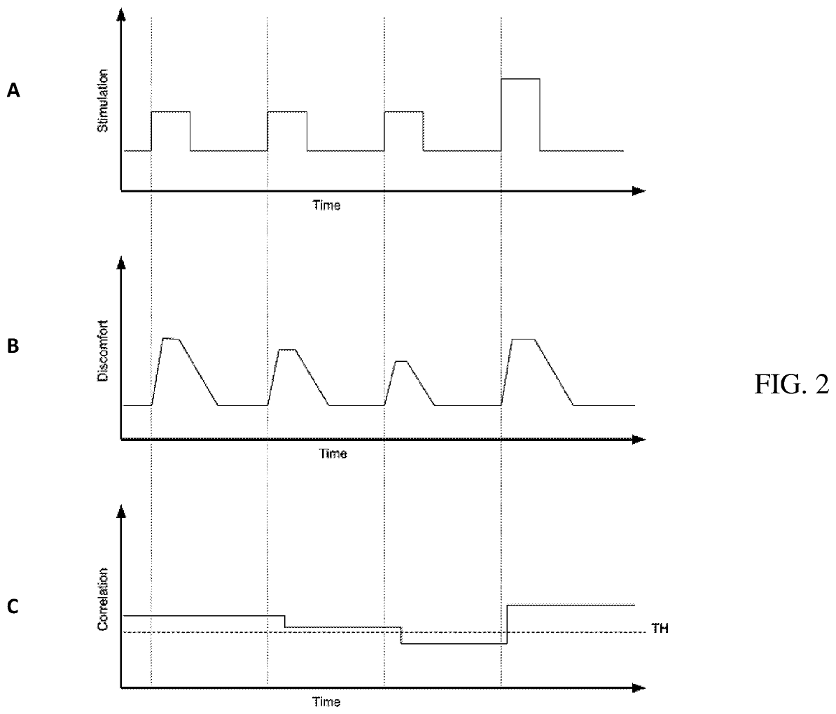

Reference is made to FIG. 2 which is a graph modeling discomfort of the user during use of the system for electrical muscle stimulation, in accordance with some applications of the present invention. Typically, a model of the discomfort of the user is constructed by measuring the correlation between the stimulation and the consequent discomfort levels. Given this correlation, control processor 204 estimates the user's discomfort levels as a result of the stimulation treatment. FIG. 2 shows that following an initial increase in user discomfort levels (B) at the start of the stimulation treatment (A), discomfort levels decrease as stimulation continues and the user adjusts to the stimulation. Given the constant stimulation, the correlation between stimulation and discomfort (C) will also decline. For some applications, as shown in FIG. 2, once the correlation declines below a certain threshold, control processor 204 allows for an increase in stimulation intensity without exceeding a predetermined limit of discomfort. Control processor 204 increases the stimulation parameters leading to an increase in discomfort of the user, causing an increase in the correlation between stimulation and discomfort, setting the cycle back into the initial phase of measuring the correlation between the stimulation and the consequent discomfort levels.

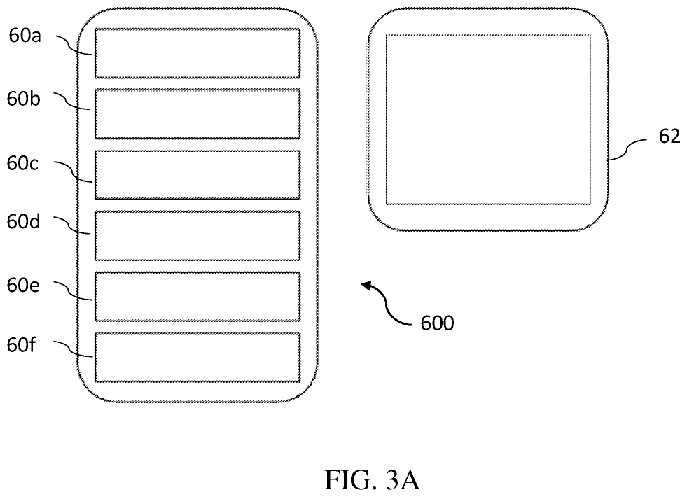

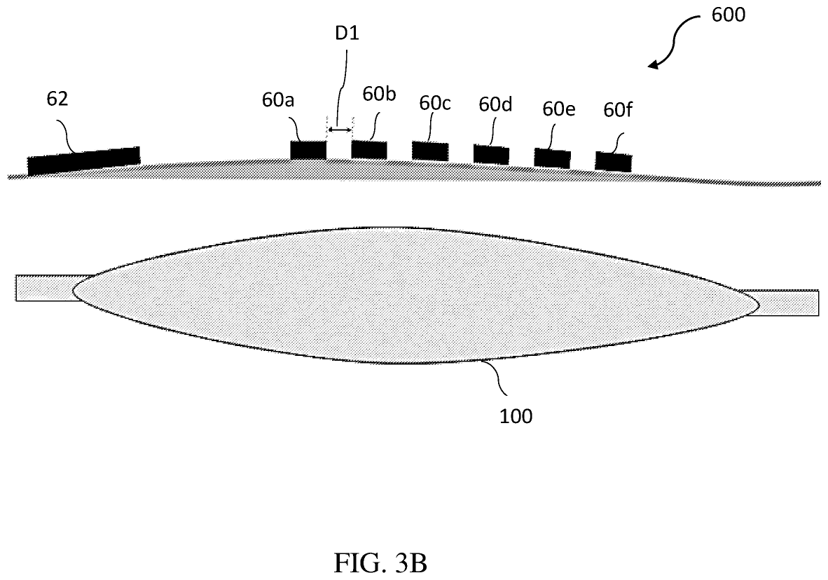

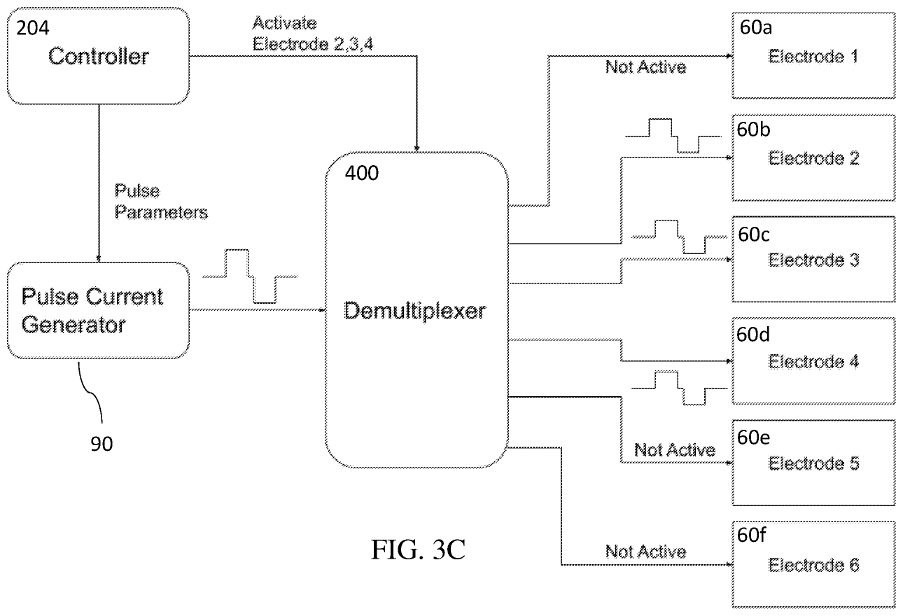

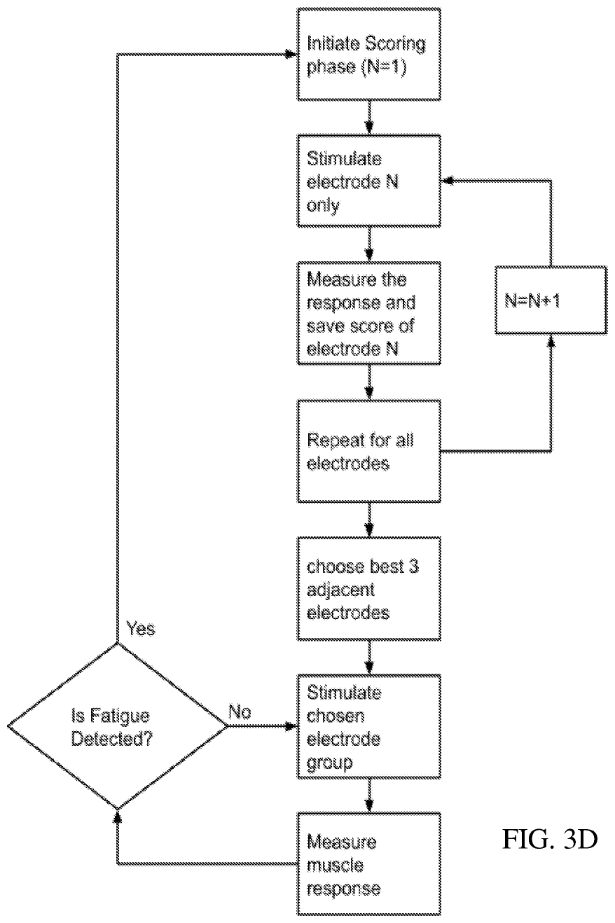

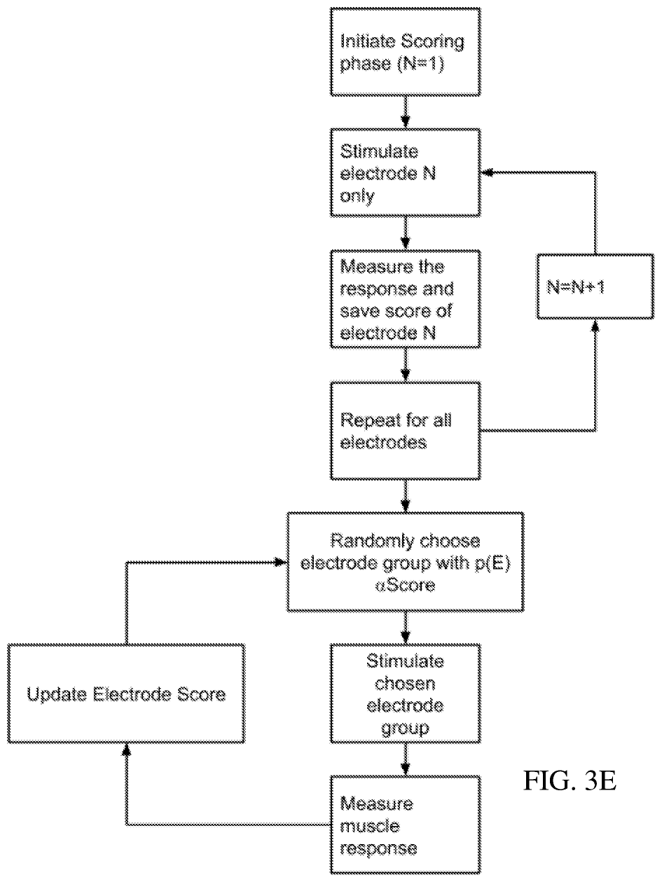

Reference is now made to FIGS. 3A-E. As described hereinabove, system 200 is configured to vary parameters of the stimulation applied to the user based on physiological parameters of the user. Such stimulation parameters may include the intensity of the stimulation, the duration of the stimulation and/or a pattern or shape of the stimulation pulses. Additionally, or alternatively, system 200 is configured to vary parameters of the stimulation applied to the user based on measuring a response of the target muscle to the stimulation. Such parameters may include a location within the target muscle tissue designated for stimulation treatment.

For example, system 200 is configured to maximize the target muscle reaction to the stimulation by controlling a location in the target muscle tissue to which the stimulation is applied. In other words, system 200 is configured, on-the-fly during a stimulation session, to change locations within the target muscle tissue to which the stimulation is applied, without physically moving the electrodes.

Reference is first made to FIGS. 3A-B, which are schematic illustration of electrode array 600 comprising plurality of stimulating electrodes 60a-60f, in accordance with some applications of the present invention. For some such applications, stimulating unit 206 comprises an electrode array 600 comprising a plurality of electrodes 60a-60f (e.g., 4-8 electrodes 60, e.g., 6 electrodes 60). When placed in the vicinity of target muscle 100, a distance D1 between each electrode is typically 2 cm or less such that electrode array 600 stimulates different portion of muscle 100 by alternating application between electrodes 60a-60f.

It is noted that array 600 is shown having six electrodes 60 by way of illustration and not limitation. Electrodes 60a-60f are spatially distributed such that the stimulation applied by each electrode reaches a portion of target muscle tissue 100 thereby facilitating selective stimulation of various portions of the target muscle tissue during a treatment session. Selective stimulation of portions of the tissue can be based on feedback received from sensing unit 202 (e.g., by measuring parameters of the muscle's response to the applied stimulation. These parameters may be indictive of muscle fatigue). Alternatively, a random one of electrodes 60a-60f (or a subset of electrodes 60a-60f) are continuously selected to apply the stimulation.

FIG. 3B shows electrode array 600 placed in proximity to muscle 100 such that each one of electrodes 60a-60f are positioned to stimulate adjacent portions of muscle 100. Typically, during a stimulation session, application of stimulation is alternated between electrodes 60a-60f (either between each electrode individually or a subset of electrodes). As noted above, switching application of the stimulation between electrodes 60a-60f is typically based on indications of muscle fatigue.

For example, at the initiation of stimulation, a first electrode, e.g., electrode 60a is activated together with electrode 62 to apply stimulation to the portion of muscle 100 that is in proximity to electrode 60a. Once muscle fatigue is detected for the portion of muscle 100 stimulated by electrode 60a, electrode 60a is deactivated and application of stimulation through electrode 60a is discontinued. Control processor 204 then selects, a second electrode, e.g., electrode 60d to be activated with electrode 62 to apply stimulation to the portion of muscle 100 that is in proximity to electrode 60d, until muscle fatigue is detected in the portion of muscle 100 that is stimulated by electrode 60d. In this manner, most, or all of electrodes 60a-60f in array 600 are activated during a treatment session to facilitate effective and uniform stimulation of muscle 100. It is noted that control unit 204 can alternate application of the stimulating current through the electrodes based on parameters other than muscle fatigue (as described elsewhere herein).

As shown in FIG. 3C, for some applications, system 200 further comprises a demultiplexer 400 for controlling selective application of stimulation to different portions of a target muscle tissue, in accordance with some applications of the present invention. Use of demultiplexer 400 allows control processor 204 to control which of electrodes 60 in array 600 are electrically coupled to pulse generator 90 to receive the pulsed current from the pulse generator to, in turn, drive current into the tissue by electrodes 60. The electrodes in array 60 that do not receive the pulse current from pulse generator 90 remain electrically floating and do not draw or drive current into the target tissue. Thus, selective stimulation of portions of the target muscle is achieved, generally without moving electrode 60.

FIG. 3C shows by way of illustration and not limitation, electrodes 60b-60d in an active state. In accordance with the description above with reference to FIG. 3B, since the electrodes in array 600 are spatially distributed, each combination of three adjacent electrodes chosen by control system 204 to apply stimulation to the tissue, stimulates a different portion of the target muscle tissue and is generally equivalent to moving the electrode to reach various locations. For some applications, array 600 is positioned such that a dimension along which the electrodes are distributed is parallel to the general direction of the muscle fibers, such that the different electrode subsets stimulate different parts of the target muscle tissue. For example, and as shown in FIG. 3C, a subset of electrodes 60b, 60c and 60d are active to stimulate muscle target tissue in the vicinity thereof, thereby controlling application of the stimulation (increase or decrease stimulation) as well as controlling the location of the stimulation without re-placement of the electrodes.

In accordance with some applications of the present invention, one or more of the following approaches can be used to control selective application of the stimulation of different portions of the muscle, and which subset of electrodes in array 600 are activated to apply stimulation to the muscle.

In a first approach, a first subset of electrodes is continuously active to stimulate a first portion of the target muscle tissue until muscle fatigue is detected in that first portion of the muscle. Once muscle fatigue is detected, electrical current through the first subset of electrodes in discontinued, and a second subset of electrodes is activated to stimulate a second portion of the target muscle tissue. For example, sensing system 202 and control processor 204 measure the muscle response and detect at least one characteristic of the stimulated target tissue. The at least one characteristic detected can be a physiological characteristic of the user, a fatigue level of a target muscle, or another characteristic related to muscle condition, performance, or reaction. For example, muscle fatigue is detected using an accelerometer which measures an amplitude of the muscle contraction. Upon detection of fatigue, system 200, adjusts at least one parameter of an application of the stimulation by discontinuing application of current through the first subset of electrodes and activating the second subset of electrodes to allow rest of the fatigued muscle portion and stimulation of a different (generally non-fatigued) muscle portion. Such measuring of the muscle response is typically conducted continuously during the treatment session. In this manner, an overall enhanced stimulation of the target muscle tissue is achieved by discontinuing stimulation to a fatigued portion of the muscle and instead applying stimulation of a non-fatigued portion of the muscle.

In a second approach, control system 204 is configured to continuously select a subset of electrodes from a random distribution that gives a higher priority to an “optimal” subset of electrodes.

In both the first and second approaches, an initial learning phase is conducted by control processor 204. Typically, this initial learning phase includes measuring the muscle response to each electrode 60 in array 600 and scoring each of the electrodes based on the response they elicit in the muscle (e.g., as determined by an accelerometer which measures an amplitude of the muscle contraction).

More specifically, in order to score an electrode, control processor 204, compares the input to the muscle (i.e., the stimulation current applied), and the muscle output (i.e., contractions of the muscle). The stimulation current is a known parameter since it originates from the system. However, the muscle contractions are usually not measured directly but rather indirectly and non-invasively. The muscle contractions (e.g., intensity of the contractions) are therefore usually measured using an accelerometer placed on the skin of the user in close proximity to the electrodes that are placed over the muscle. In order to estimate the muscle's reaction to the applied stimulation, control processor 204 compares the energy measured by the accelerometer at two time periods: (a) the acceleration signal energy measured in the 2 second time period prior to application of stimulation; and (b) the acceleration signal energy measured in the 0.5 second subsequent to the initiation of the stimulation.

Using the following formula (T=Time when stimulation started; Stimulation=total stimulation current in the first 0.5 seconds of the stimulation burst; ACC(t)=the acceleration signal at time t):

$${ACC}_{energy - Base} = {\sum }_{t = T - 2}^{t = T} {\vert {ACC}_{( t )} \vert}^{2} \\ {ACC}_{energy - Reaction} = {\sum }_{t = T}^{t = T + 0.5} {\vert {ACC}_{( t )} \vert}^{2} .$$

Using the two above ACC values the Normalized Reaction can be defined using the following formula:

$$Normalized Reaction = \frac{4 \cdot {ACC}_{energy - Reaction}}{{ACC}_{energy - Base}} \cdot \frac{1}{Stimulation} .$$

In order to obtain the score for a single electrode in array 600, the values for the current driven through this single electrode is used only in the calculation of the stimulation.

Reference is now made to FIGS. 3D-3E, which are flow charts representing the first (FIG. 3D) and the second (FIG. 3E) algorithm-based approaches for controlling selective application of the stimulation to different portions of the muscle, by selected subsets of electrodes in the electrode array, in accordance with some applications of the present invention.

As shown in FIG. 3D, in the first approach, control system 204 selects a first subset of electrodes based on scores (as described hereinabove). Stimulation to the portion of the muscle tissue is then applied by the first group and the muscle response is continuously monitored. Once a change in the response is detected and identified by control processor 204 as muscle fatigue, the control processor performs another learning cycle to identify a second optimal subset of electrodes. Re-scoring of the electrodes is done, and a second subset of electrodes are selected to stimulate a different portion of the target muscle tissue.

In accordance with some applications of the present invention, muscle fatigue is detected by indirect acceleration measurements using an accelerometer placed on the skin of the user in close proximity to the electrodes that are placed over the muscle, as described herein. In accordance with some applications of the present invention, muscle fatigue is defined as a significant decline in the response of the muscle to the stimulation (the response measured by muscle contractions detected by the accelerometer), as follows:

$$Normalized Reaction = \frac{4 \cdot {ACC}_{energy - Reaction}}{{ACC}_{energy - Base}} \cdot \frac{1}{Stimulation} .$$

In order to reduce the effect of noise (e.g., caused by body signals that are generally not related to the muscle stimulation), the muscle reaction to multiple stimulation bursts is compared to examine whether significant changes have occurred (e.g., over multiple bursts, e.g., 10).
Reaction(n) is the normalized reaction of a burst number n (e.g., n=1, 2, 3 . . . 10):
R̅e̅a̅c̅t̅i̅o̅n̅₁̅₀̅(̅n̅)̅=the mean of the normalized reaction in the 10 bursts of stimulation previous to n.
σ_{Reaction}_{₁₀}(n)=the standard deviation of the normalized reaction in the 10 bursts previous to n(n>10).

$$Reaction Change ( n ) = \frac{Reaction ( n ) - \overline{{Reaction}_{10} ( n )}}{{σ}_{{Reaction}_{10}} ( n )} .$$

The electrode-muscle reaction is defined as fatigue if the Reaction change is under (−1).

Reference is now made to FIG. 3E which is a flow chart demonstrating the second approach for scoring and selecting electrodes 60 in array 600, in accordance with some applications of the present invention. As shown in FIG. 3E, in the second approach the electrodes are scored in the initial learning phase, followed by selection of a random subset of electrodes 60 from the spatial distribution of the electrodes in array 600. In other words, the subset of electrodes is selected based on high scoring in the learning phase.

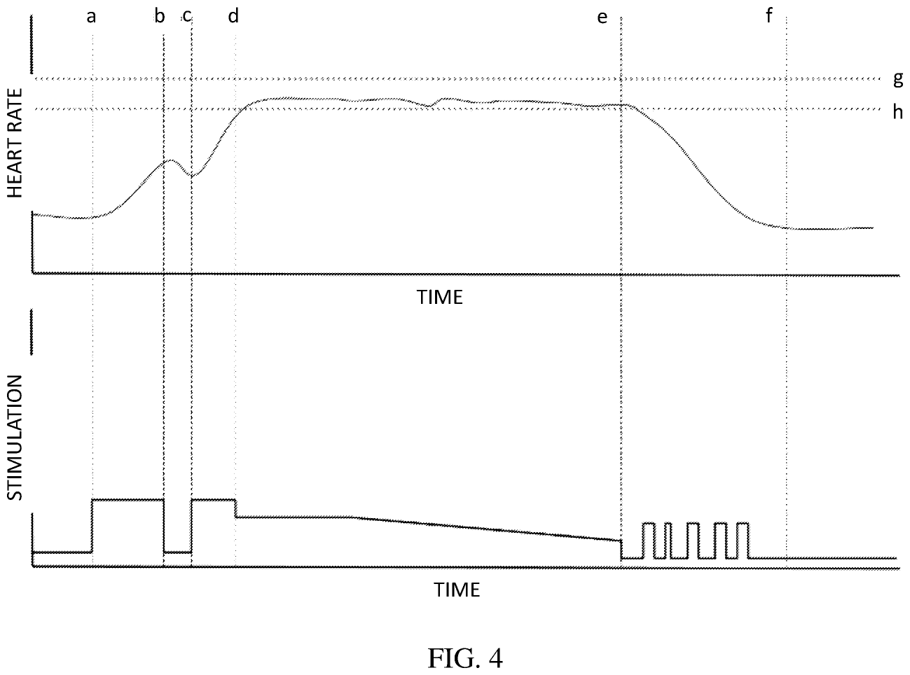

Reference is now made to FIG. 4. In some applications, system 200 is configured to apply electrical stimulation for cardiac training and rehabilitation in sedentary patients. For some such applications system 200 is configured to assists elderly or disabled patients perform light to mild cardiovascular workouts as prescribed by a physician by raising the patient's heart rate (HR) to a pre-determined level and maintain the heart rate at the desired level for the duration of the stimulation treatment session. Typically, stimulation is applied to muscles in the legs and the gluteal muscles. Stimulation of the gluteal muscles and muscles in the legs by system 200 elevates the HR in a controlled manner while the patient's HR and discomfort levels are continuously monitored by sensing system 202 and control system 204 to ensure safety of the stimulation treatment. System 200 is configured to gradually increase intensity of the applied stimulation to the muscles to gradually increase the HR until a predefined HR goal is reached. Typically, for such applications, system 200 comprises sensing unit 202, which is placed across a chest of the patient and configured to measure an ECG signal of the patient and stimulating unit 206 comprising electrodes which are placed on buttocks and quadriceps of the user. Control processor 204 is configured to process the signal from sensing unit 202 and instruct stimulating unit 206 to drive current into the patient in a controlled manner such that the HR of the patient is elevated beyond a first predetermined level but at the same time does not exceed a second, higher, predetermined level. Thus, system 200 is configured to raise the patient's HR to a rate that is both safe and beneficial.

As shown in FIG. 4, for some applications, the physician may define two HR levels: (a) a first level being a first low threshold (indicated by “h” in FIG. 4), in which the HR that is reached during a treatment session should be higher than this first level of the low predefined threshold; and (b) a second level being a high threshold (indicated by “g” in FIG. 4). The second threshold marks a HR rate that must never be exceeded.

An example of a training session of five stages, in accordance with some applications, is described as following:
Signal checks: Following placement of sensing unit 202 and stimulation unit 206, control processor 204 processes the signal obtained from the sensors in sensing unit 202 and calibrates the system. Once the sensors (e.g. ECG sensors) display a desired signal, control processor 204 records the signal from the sensors (e.g., for a duration of 60 seconds), and processes the ECG signal to determine whether the patient exhibits signs of abnormal cardiac activity (e.g., arrhythmia). In cases in which an abnormality is detected by system 200, an error message is displayed on user interface 205 and the operation of the treatment session is discontinued.
Stimulation exploration: Each electrode (or stimulation channel of two or more electrodes) is stimulated and the response from each location stimulated is recorded by system 200. Once the optimal subset of electrodes in the array is selected (as described herein with reference to FIGS. 3A-E), control processor 204 begins with a low intensity stimulation of the quadriceps muscles while monitoring the effect of the simulation on the patient's HR and discomfort (stress) levels. Once a desired effect of the quadriceps muscles is established (e.g., as measured by an increase in HR), system 200 moves on to measure the effect of the stimulation on the glutes (e.g., as measured by an increase in HR). In cases in which an extreme increase in discomfort levels is observed or if an abnormality in cardiac activity is detected, stimulation is immediately discontinued.
Heart rate increase: In this stage, control system 204 starts (1) to stimulate the gluteal muscles and quadriceps muscles (either together or separately) in order to gradually increase the HR of the patient to the predetermined low threshold (as described herein above). The control processor learns the relationship between the applied stimulation and the patient's cardiovascular response, and the effect of the stimulation on the HR of the patient is updated and recorded by control processor 204. Subsequently to the HR reaching at least 75% of the first low predetermined threshold, (2) system 200 stops stimulation and records the cardiovascular response as assessed by the decrease in HR. The decrease in HR is typically indictive of the patient's cardiovascular fitness and is used to correctly estimate the patients' cardiac response to the stimulation. Once the decrease in the HR is determined, (3) control system 204 resumes stimulation of the muscles for HR elevation.
Stabilizing elevated HR: Prior to the patient's HR reaching the first (lower) predetermined HR level (4) control system 204 initiates a decrease in the rate and the intensity of the stimulation applied to the patient, in order to dampen the increase in the HR of the patient. Muscle stimulation is linked to the second derivative of the HR and in order to avoid overshooting the second (higher) HR threshold, control system 204 lowers the intensity of stimulation prior to reaching the first (lower) HR threshold. The exact timing and rate of decrease generally depends on the measured increase in the HR during stimulation and decease thereof in the absence of stimulation. For some applications, control processor 204 generally aims to maintain the patient's HR just above (5 bpm) the lower threshold for a predetermined duration. The rate of simulation is corrected in real time as it is likely that with cardiovascular fatigue the HR will continuously creep up if the stimulation level is kept constant.
Cooldown: Subsequently to reaching the predetermined duration of the stimulation (5), control processor 204 reduces the intensity of the stimulation allowing the HR to decrease. During the cooldown stage the stimulation focuses on increasing perfusion to the activated muscles in order to reduce the level of delayed onset of muscle soreness. When the patient's HR returns to baseline values, (6) the treatment session is terminated. The session statistics are recorded in system 200. In the next stimulation treatment session control processor 204 uses the recorded data to create the models for control prior to initiation of the session. These models are updated during the session saved and re-used during the following session.

For some applications, system 200 is configured to synchronize heart beats with stimulation of the gastrocnemius and soleus muscles, thereby increasing blood perfusion to the foot to reduce the risk of diabetic foot symptoms. It is hypothesized by the inventors that contractions of calf muscles (in particular the gastrocnemius and soleus muscles) acts as a peristaltic pump and has an effect of assisting pumping of blood from the heart to the foot. Patients suffering from Peripheral Artery Disease (PAD) suffer from a decreased perfusion to the foot and consequently may suffer from several conditions such as chronic ulcers.

In order to assist these patients, control processor 204 uses a closed loop control to increase perfusion to the leg by synchronizing stimulation of the muscle with the heart beat phase and additionally ensure that the increase in pressure in the blood vessels occurs at an optimal time with respect to the cardiac cycle (e.g., at the systolic phase)

For some applications, control processor 204 operates in an open loop manner by synchronizing application of the stimulation to the signal obtained from the ECG sensor or alternatively can be applied in a closed loop manner by adding an input sensor that measures the perfusion to the foot either directly or indirectly (e.g., direct measurements may include measuring blood flow using ultrasound doppler or any other method for measuring blood flow and indirect measurements may include measuring oxygen levels, blood pressure, skin coloration, and/or skin temperature). One such option is to add a PPG sensor. By analyzing the morphology of the heart-rate signal measured, system 200 could derive a perfusion index and stimulate the muscle as needed to increase the index to a desired level.

Reference is again made to FIGS. 1-4. It is noted that system 200 may be used to stimulate any target muscle or a portion of a muscle in the user's body. Additionally, or alternatively, system 200 may be used to stimulate two or more muscles simultaneously. The electrical stimulation may be applied to the muscles in accordance with various treatment protocols. An example of such a protocol for EMS treatment may include: placing a first group of 3 electrodes (e.g., electrodes A, B and C) on the same side of the abdominal rectus muscle and a second group of electrodes (e.g., comprising at least electrode D) on the external abdominal oblique muscle. According to this example the following sequence can be applied: (a) Ramp up stimulation on A&B electrodes, (b) Muscle “C” is in passive strain, (c) while holding on A&B electrodes, start a ramp on C&D electrodes, (d) releasing A&D electrodes without ramp down, (e) muscle “A” is in passive strain, (f) start with ramp up on A&D, (g) continue as same for B and C. It is noted that any other suitable stimulation protocol may be applied.

In some embodiments, the present disclosure provides for a method for automated, continuous, real-time adjusting of EMS parameters, based, at least in part on continuous measuring of muscle movement.

In some embodiments, the present disclosure provides for placing a plurality of movement detection sensors, e.g., accelerometers, about a muscle stimulation region of a user of the present system. In some embodiments, the movement detection sensors are configured to measure acceleration, e.g., the rate of change of velocity, in one or more degrees-of-freedom.

In one embodiment, a sensing unit, e.g., stimulating unit 206 of electrical muscle stimulation system 200 in FIG. 1, may be configured to comprise one or more accelerometers within sensors module 30, to measure and monitor muscle reaction during a treatment session. More specifically, the accelerometers device may be operable to detect an onset, a modification, or a disappearance of a muscle response in reaction to a stimulation signal applied to the muscle by stimulating unit 206. In other embodiments, sensors module 30 of stimulating unit 206 may comprise any other suitable sensors, e.g., tension strain gauges.

In some embodiments, when a stimulation signal is delivered to a muscle, the muscle will react to some degree and a physical movement response will be registered by the accelerometers. In some embodiments, the accelerometer may be configured to detect successive muscle reaction pulses in response to, e.g., pulse signals delivered to the muscle.

In some embodiments, the movement detection sensors are disposed about an electrode array of the present disclosure, e.g., electrode array 600 in FIG. 3A. In some embodiments, electrode array 600 may be configured for application in the vicinity of a target muscle, e.g., target muscle 100 in FIG. 3B to stimulate one or more regions of muscle 100. In some embodiments,

In some embodiments, the movement detection sensors are arranged about the electrode array 600 in a predetermined pattern in relation to a center point or region of the muscle. In some embodiments, the predetermined pattern is configured to measure muscle movement and/or acceleration at its mid-point. In some embodiments, the signals received from the plurality of movement detection sensors may be aggregated, e.g., based on a weighting of the signal received from each movement detection sensor, based on its geometric distance from the muscle mid-point or region.

In some embodiments, the aggregated signals received from the plurality of movement detection sensors may be used to determine a normalized muscle response to a specified EMS signal. In some embodiments, normalized muscle response represents the extent to which a muscle moves relative to the specified provided stimulation energy. In some embodiments, normalized muscle response may be calculated by determining a reference muscle response, defined as muscle movement immediately before (e.g., 0.5 seconds before) application of a stimulating signal, and a stimulated muscle response defined as muscle movement immediately after (e.g., 0.5 seconds after) application of a stimulating signal. In some embodiments, normalized muscle response may be given as:

Normalizedresponse=(responseenergy−refenergy)/(stimulationenergy).

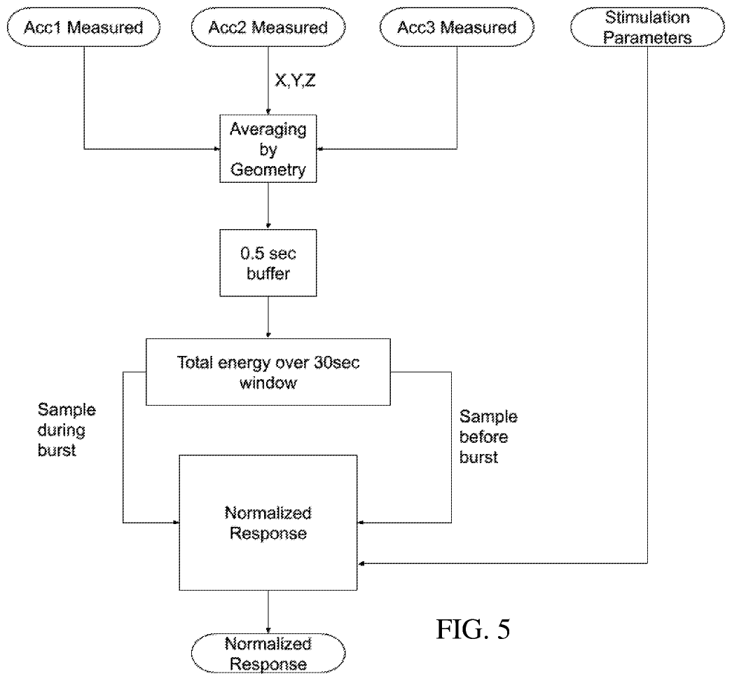

FIG. 5 is a schematic diagram of the functional steps in a process of the present disclosure for calculating normalized muscle response to known EMS stimulation.

In some embodiments, the present disclosure provides for an algorithm for muscle fatigue estimation, based, at least in part, on calculated normalized muscle response to a stimulation signal.

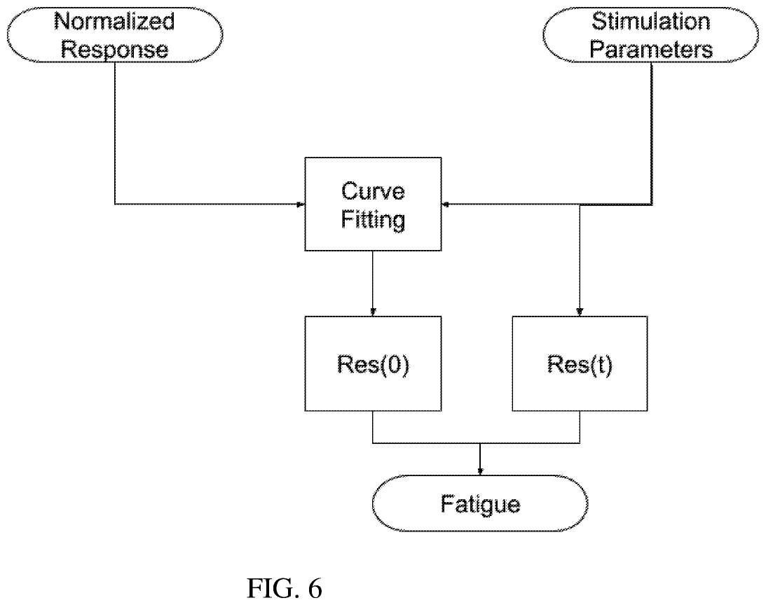

FIG. 6 is a schematic diagram of the functional steps in a process of the present disclosure for muscle fatigue estimation. In some embodiments, the present algorithm calculates a fatigue score representing a rate of fatigue onset in the muscle, in response to a stimulation signal. A fatigue level experienced by a muscle varies and generally accelerates as treatment progresses. Fatigue levels experienced and the acceleration of fatigue will be different for each user and each target muscle or group and must be carefully monitored to increase the efficiency of electro-stimulation in sports applications. The measurement of a degree of muscular fatigue is an indication to a user continuing muscle stimulation during a particular treatment session offers no further benefit.

In some embodiments, a fatigue score of the present disclosure is based on a ratio between a reference muscle response and a muscle response at a time t during stimulation treatment session.

In some embodiments, a fatigue score of the present disclosure is based on a response decay model defined as:

$$\frac{d ( Res ( t ) )}{dt} = - k \frac{Res ( t )}{Res ( t = 0 )} {E}_{stim} ( t ) + R ( Res ( t = 0 ) - Res ( t ) )$$

where Res(t) is the normalized response at time t and Eₛₜᵢₘ is the stimulation energy at time (t). K is a fitness coefficient, wherein a lower value generally denotes better muscle resistance to fatigue. R is a recovery coefficient. The fatigue score in this case will also be the ratio between the current response and the initial response, but the initial response will be calculated by fitting the stimulation energy and the measured response to the model and estimating Res(t=0).

In some embodiments, the present disclosure provides for estimation a fitness score with respect to a user based, at least in part, on a history of measured normalized muscle response in the user, wherein K is a fitness coefficient which may be derived from the equation provided immediately above.

In some embodiments, the present disclosure provides for determining a treatment protocol for a user, which comprises a succession of treatment segments representing varying stimulation levels, alternating between, e.g., high intensity, medium intensity, low intensity, and/or rest segments. In some embodiments, each segment may be configured to exercise and stimulate a muscle to a specified level of muscle fatigue, wherein a fatigue level is generally directly correlated with an intensity level represented by a segment. In some embodiments, rest segments may be configured to provide very low intensity stimulation, or no stimulation at all, to permit the muscle to recuperate and return to a lower fatigue level.

In some embodiments, the present disclosure may provide for an automated dynamically-adjusted treatment protocol comprising a succession of treatment segments representing varying stimulation levels, alternating between, e.g., high intensity, medium intensity, low intensity, and/or rest segment. In some embodiments, an electrical muscle stimulation system of the present disclosure, e.g., electrical muscle stimulation system 200 in FIG. 1, may automatically generate a treatment protocol of the present disclosure for a specified user, based, at least in part, on an estimated fitness score of the user. In some embodiments, electrical muscle stimulation system 200 in FIG. 1 may automatically dynamically adjust a provided template treatment protocol of the present disclosure for a specified user, based, at least in part, on an estimated fitness score of the user, wherein the provided template protocol defines initial segment durations, the stimulation parameters per segment, and a muscle fatigue threshold per segment.

In some embodiments, electrical muscle stimulation system 200 of the present disclosure may be put to use with respect to a specified user. System 200 may continuously dynamically adjust parameters of the treatment protocol, e.g., by estimating a fatigue score for the user, as detailed hereinabove, and adjusting treatment segment parameters to reach a specified predetermined fatigue level at the end of a segment of the segments. In some embodiments, a degree of adjustment or modification of a template protocol may be limited, e.g., to a maximum of 30%, e.g., between 20-40% increase in a modified parameter, e.g., segment intensity and/or duration.

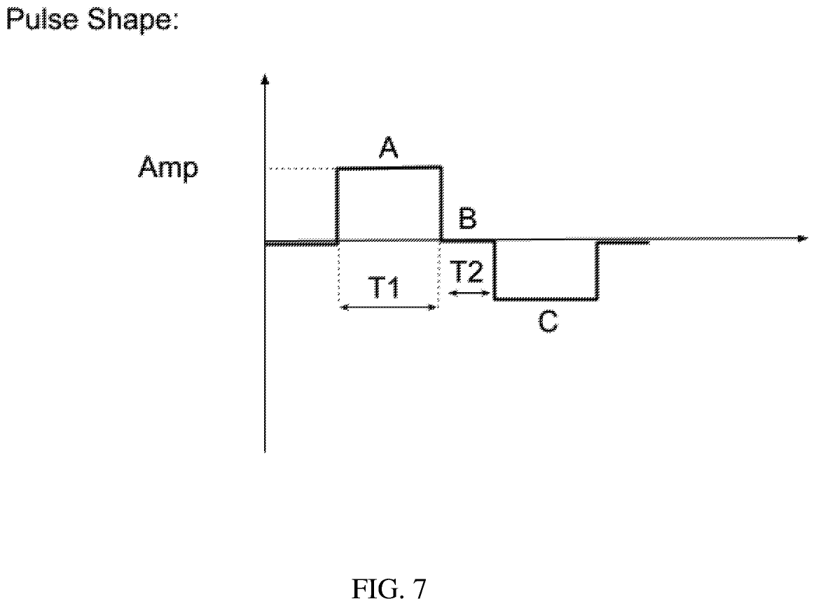

Reference is made to FIG. 7 which illustrates an exemplary treatment signal pulse shape of the present disclosure. In some embodiments, the present disclosure provides for a symmetric bi-phasic rectangular pulse shape comprising two rectangular phases, one positive (A) and the other negative (C). The two phases are of the same area, to avoid accumulation of charge on the patient's skin. In some embodiments, a length of the pulse phases (A, C) is in the range of 100-400 microseconds (p). In some embodiments, a length of an interphasic rest period is 40-100 μs. In some embodiments, a pulse of the present disclosure may be current controlled and has an amplitude of 20-100 mA peak-to-peak. In some embodiments, pulses may be provided in bursts of 20-70 Hz, for a duration of 0.5-15 seconds.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A system for stimulating a muscle of a user, the system comprising:
a stimulating unit comprising (i) at least two electrodes configured to be placed in electrical contact with skin of a user in a vicinity of the muscle, and (ii) a pulse generator configured to generate an electrical pulse for application to the muscle through the at least two electrodes;
a sensing unit comprising at least one sensor configured to measure a physiological parameter of the user and generate a sensing signal in response thereto; and
a control processor operatively coupled to the stimulating unit and to the sensing unit and configured to receive the sensing signal from the at least one sensor and change a parameter of the electrical pulse applied by the at least two electrodes in response to the sensing signal.

2. The system according to claim 1, wherein the parameter of the electrical pulse is one or more of: an intensity of the pulse, a duration of the pulse, and a shape of the pulse, or a combination thereof.

3. The system according to claim 1, wherein said electrical pulse comprises a symmetric bi-phasic rectangular pulse shape comprising equal positive and negative pulse phases.

4. The system of claim 3, wherein (i) a length of each of said pulse phases is in the range of 100-400 microseconds (μs), (ii) a length of an interphasic rest period is within the range of 40-100 μs, (ii) an amplitude of each of said pulse phases is in the range of 20-100 mA.

5. The system of claim 1, wherein said applied electrical pulse-comprises pulse bursts having a frequency of between 20-70 Hz and a duration of between 0.5-15 seconds.

6. The system according to claim 1, wherein the physiological parameter of the user includes a parameter selected from the group consisting of: body movements, body position, heart rate (HR), heart rate variability (HRV), oxygen saturation, and a rate of respiration, and wherein the at least one sensor is configured to measure the physiological parameter selected from the group.

7. The system according to claim 1, wherein the sensing unit comprises a breathing sensor configured to detect breathing of the user and wherein the control processor is configured to change the parameter of the electrical pulse in response to identifying a phase of the breathing cycle based on the sensing signal.

8. The system according to claim 1, wherein the sensing unit comprises a Galvanic Skin Response (GSR) sensor and wherein the control processor is configured to determine a level of discomfort of the user based on the sensing signal from the GSR sensor, and calculate a correlation between the electrical pulse applied by the stimulating unit and the level of discomfort of the user and to change the parameter of the electrical pulse based on the correlation.

9. The system according to claim 1, wherein the sensing unit comprises an accelerometer and wherein the physiological parameter of the user comprises contractions of the muscles measured using the accelerometer.

10. The system according to claim 1, wherein
the stimulating unit is configured to provide the electrical muscle stimulation such that contractions of the stimulated muscle exceed a desired threshold;
and wherein,
the control processor is configured to change the parameters of the pulse applied to the muscle in order to exceed the desired threshold of contractions, based on multiple uses of the system and:
(i) iteratively varying the parameters of the electrical pulses, and
(ii) measuring an effect of the varying of the parameters of the electrical pulses on the muscle contractions.

11. (canceled)

12. (canceled)

13. (canceled)

14. The system according to claim 1, further comprising at least one of: (i) a heat source configured to heat the skin in proximity of the muscle to be stimulated by up to 20 degrees Celsius, (ii) a vacuum source configured to apply vacuum to the skin of the user in proximity of the muscle to be stimulated, and (iii) at least two accelerometers arranged in a predetermined pattern in relation to a center point of the muscle, and wherein each of said accelerometers is configured to measure movement of said muscle in one or more degrees-of-freedom.

15. (canceled)

16. (canceled)

17. The system of claim 14, wherein said movement comprises at least one of: an onset, a modification, and a disappearance of a physical movement at said center point in reaction to said electrical pulse.

18. (canceled)

19. (canceled)

20. (canceled)

21. (canceled)

22. (canceled)

23. (canceled)

24. (canceled)

25. A method for electrically stimulating a muscle of a user, the method comprising:
placing first and second electrodes in contact with skin in a vicinity of the muscle;
stimulating a first portion of the muscle by applying electrical pulses to the muscle through the first electrode.
using a sensor, sensing a muscle response, the muscle response being indictive of a parameter of the muscle response of the first muscle portion;
based on the sensing:
discontinuing application of the electrical pulses to the muscle through the first electrode, and
stimulating a second portion of the muscle by applying electrical pulses to the muscle through the second electrode.

26. The method according to claim 25, wherein sensing a muscle response comprises sensing a parameter of contractions of the muscle indicative of muscle fatigue.

27. The method according to claim 26, wherein the parameter of the contractions includes an intensity of the contractions and wherein the sensing comprises sensing the intensity of the contractions.

28. (canceled)

29. A method for electrically stimulating a muscle of a user, the method comprising:
placing a stimulating unit comprising (i) at least two electrodes configured to be placed in electrical contact with skin of a user in a vicinity of the muscle, and (ii) at least two accelerometers arranged in a predetermined pattern in relation to a center point of the muscle, wherein each of said accelerometers is configured to measure movement of said muscle in one or more degrees-of-freedom;
operating a pulse generator to generate an electrical pulse for application to the muscle through the at least two electrodes;
measuring, through said at least two accelerometers, movement of said muscle at said center point in response to said application; and
adjusting, by a control processor operatively coupled to the stimulating unit and the pulse generator, at least one parameter of said electrical pulse, based, at least in part, on said measuring, wherein the parameter of the electrical pulse is one or more of: an intensity of the pulse, a duration of the pulse, and a shape of the pulse, or a combination thereof.

30. (canceled)

31. The method of claim 29, further comprising calculating, by said control processor, a normalized response value of said muscle to said electrical pulse, wherein said normalized response value represents a ratio between said movement of said muscle and a reference movement of said muscle before said application, and estimating, by said control processor, a fatigue score of said muscle, wherein said fatigue score is based, at least in part, on said normalized response value calculated over time.

32. (canceled)

33. The method of claim 31, further comprising modifying, by said control processor, a treatment protocol for a user, wherein said treatment protocol comprises a succession of electrical pulse applications, and wherein each of said applications is configured to generate a specified level of fatigue in said muscle, based, at least in part, on said fatigue score, wherein said modifying comprises modifying at least one of: an intensity of said electrical pulse application, a duration of said electrical pulse application, and a shape of a pulse signal of said electrical pulse.

34. (canceled)

35. The method according to claim 29, further comprising sensing at least one physiological parameter of the user through a sensing unit comprising at least one sensor, wherein the physiological parameter of the user includes a parameter selected from the group consisting of: body movements, body position, heart rate (HR) heart rate variability (HRV), oxygen saturation, and a rate of respiration, and wherein the at least one sensor is configured to measure the physiological parameter selected from the group.

36. (canceled)

37. The method according to claim 35, wherein the at least one sensor comprises at least one of: (i) a sensor configured to detect breathing of the user, and wherein the control processor is configured to perform said adjusting, at least in part, in response to identifying a phase of a breathing cycle of the user, and (ii) a Galvanic Skin Response (GSR) sensor, and wherein the control processor is configured to determine a level of discomfort of the user based on the sensing signal from the GSR sensor, and calculate a correlation between the electrical pulse applied by the stimulating unit and the level of discomfort of the user and to perform said adjusting, at least in part, based on the correlation.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)
